(19) United States
(12) Patent Application Publication
(10) Pub. No.: US 2003/0118658 A1
(43) Pub. Date: Jun. 26, 2003

(54) High aspect ratio encapsulated inorganic antimicrobial additive for controlled release

(76) Inventors: Jeffrey A. Trogolo, Boston, MA (US); Frank C. Rossitto, Danvers, MA (US); Edward K. Welch II, Ipswich, MA (US)

Correspondence Address: AGION TECHNOLOGIES, 60 Audubon Road, Wakefield, MA, 01880, US

(21) Appl. No.: 10/032,370

(22) Filed: Dec. 21, 2001

(51) Int. Cl. A61K 33/42; A61K 33/38; A61K 33/32; A61K 33/34; A61K 9/16

(52) U.S. Cl. 424/490; 424/618; 424/604; 424/630; 424/641

(57) ABSTRACT

The invention relates to a high aspect ratio microcapsule comprising an inorganic antimicrobial agent coated with a hydrophilic polymer. The hydrophilic polymer is able to absorb sufficient water as to enable the action of the encapsulated antimicrobial agent. These high aspect ratio microcapsules are useful to impart antimicrobial activity and can be used in polymer compositions, sprays and coatings. A method of preparing the high aspect ratio microcapsule by melt compounding and fabrication of the antimicrobial agent with the hydrophilic polymer is provided. Polymer compositions comprising the high aspect ratio microcapsules and a matrix polymer are also provided. Another embodiment of the invention is an article prepared from the polymer compositions comprising the high aspect ratio microcapsules and a matrix polymer.

### FIELD OF THE INVENTION

This invention relates antimicrobial agents that are encapsulated with a hydrophilic coating and formed into high aspect ratio microcapsules.

### BACKGROUND OF THE INVENTION

A number of inorganic materials have been shown to possess antimicrobial activity. They include metal ions such as silver, copper, zinc, mercury, tin, lead, bismuth, cadmium, chromium and thallium ions. It is theorized that these antimicrobial metal ions exert their effects by disrupting respiration and electron transport systems upon absorption into bacterial or fungal cells. Antimicrobial metal ions of silver, copper, zinc, and gold, in particular, are considered safe for in vivo use. Antimicrobial silver ions are particularly useful for in vivo uses due to the fact that they have the highest ratio of efficacy to toxicity.

Antimicrobial zeolites can be prepared by replacing all or part of the ion-exchangeable ions in zeolite with antimicrobial metal ions, as described in U.S. Pat. Nos. 4,911,898; 4,911,899; 4,938,955; 4,906,464; and 4,775,585.

Zirconium compounds, such as zirconium phosphates, have also been modified to provide antimicrobial characteristics, as described in U.S. Pat. Nos. 4,025,608 and 4,059,679. J. Antibact. Antifung. Agents Vol. 22, No. 10, pp. 595-601, 1994 and references therein describe the antimicrobial characteristics of zirconium phosphate ceramics.

Antimicrobial water soluble glasses have been used and are described in U.S. Pat. No. 5,470,585.

Antimicrobial hydroxyapatite powders have been prepared and are described in U.S. Pat. Nos. 5,009,898 and 5,268,174.

U.S. Pat. No. 4,775,585 discloses incorporating metal-zeolite into a polymer to obtain a polymer with bactericidal activity. U.S. Pat. No. 4,923,450 discloses incorporating zeolite in bulk materials for production of medical tubes. Dependent upon material selection and processing conditions, when zeolite is conventionally compounded into polymers, the zeolite often agglomerates, causing poor dispersion of the zeolite in the polymer. When such material is molded or extruded, the surface of the polymer is frequently beaded instead of flat. Poor dispersion of the zeolite also can cause changes in the bulk properties of the polymer, such as a reduction in tensile strength.

Furthermore, it has been found by the present inventors that conventionally compounding antimicrobial zeolites in many polymeric materials, dependent upon processing conditions and the particular polymer, can result in discoloration. This appears also to result from inadequate dispersion of the zeolite, i.e., the formation of zeolite aggregates in the material as well as from chemical reactions involving the antimicrobial metal ions and the polymer itself, e.g., additives, contaminants, residual catalysts, moisture, etc. in the polymer and/or any air or water introduced during the compounding process.

In certain instances, these problems can be avoided by use of antimicrobial coatings. However, this requires an extra processing step and raises additional problems such as adherence and permanence of the coating.

U.S. Pat. No. 5,094,847 recognizes that in order to get the desired antibacterial activity, a large amount of zeolite powder must be added to the polyolefin resin and that this is accompanied by poorer appearance, lower physical properties and roughened surface appearance. They disclose using low levels of antimicrobial agent followed by a corona discharge treatment. This requires additional equipment and an extra processing step.

U.S. Pat. No. 5,614,568 discloses an antibacterial resin composition comprising a styrene resin, an antibacterial agent and a compound or polymer having at least one functional group and a molecular weight of 300 to 10,000. In order to obtain good antimicrobial activity, they require the use of low molecular weight compounds or low molecular weight polymers, which can have deleterious effects on properties.

U.S. Pat. No. 6,013,275 discloses a copolymer of an antibacterial agent and a hydrophilic substance. Copolymerization is an extra step, which adds to complexity and cost and limits the choice of both the hydrophilic substance and the antibacterial agent. The hydrophilic substance must have functional groups capable of reacting with and copolymerizing with the antibacterial agent. Similarly, the antibacterial agent must have reactive groups capable of forming a copolymer with the antibacterial agent.

WO 00/30697 discloses an antimicrobial coated substrate comprising an antimicrobial coating composition coated on a substrate. The antimicrobial coating composition comprises a hydrophilic polymer having antimicrobial ceramic particles dispersed therein. They require a coating process. This requires additional equipment and an extra processing step.

One of the problems in the prior art is the unavailability, for the most part, of that quantity of the antimicrobial agent which lies beneath the surface of the article or coating into which it is incorporated. Unless the antimicrobial agent migrates from the polymer matrix, a characteristic not common to inorganic, especially ion exchange type, antimicrobial agents, the entombed antimicrobial agent is without utility or efficacy. This requires the use of a larger quantity of antimicrobial agents so as to provide a higher concentration at the surface, which is more costly and often imparts deleterious properties. There remains a need to provide an antimicrobial agent in a form that is suitable to impart antimicrobial properties without the accompanying problems of the prior art.

Another problem in the prior art is that while the use of hydrophilic coatings and polymer matrices may mitigate, at least in part, the foregoing problem, this beneficial improvement is limited to utility in the narrow class of hydrophilic coatings and polymers and, more importantly, the very limited end use applications for which such hydrophilic coatings and polymers are appropriate. Thus, there is also a need in the art to find a means by which antimicrobial agent entombed within a polymer coating or matrix can be accessed or available for providing antimicrobial efficacy regardless of polymer comprising the coating or matrix.

Yet another problem in the prior art is that the rate of release of the antimicrobial agent is determined by the solubility of the antimicrobial agent or, in the case of the ion-exchange type antimicrobial agents, the ion-exchange rate of the ceramic carrier and the water exposure or flow across the surface containing the antimicrobial agent. In high moisture, especially high flow environments, for example, a dishwasher interior, a high solubility or ion-exchange rate may lead to a premature depletion of the antimicrobial agent; thus, greatly reducing the life time of the antimicrobial efficacy. Thus, there is also a need in the art to be able to control the release rate of the antimicrobial agent.

### SUMMARY OF THE INVENTION

This invention provides for high aspect ratio microcapsules. These high aspect ratio microcapsules comprise an antimicrobial agent, typically in the form of a particle or particles, encapsulated within a hydrophilic polymer and fabricated to form high aspect ratio microcapsules. The fabrication can be achieved by any of several methods known in the art for manufacturing shaped particles including melt spinning to form fibers and melt casting followed by flaking to form flaked particles. The hydrophilic polymer is able to absorb sufficient water as to enable the action of the encapsulated antimicrobial agent. These high aspect ratio microcapsules are useful to impart antimicrobial activity and can be used in polymer compositions, sprays and coatings.

Also a method is provided for preparation of the high aspect ratio microcapsule by compounding of the antimicrobial agent with the hydrophilic polymer and subsequently flaking or spinning the compounded product.

Yet another embodiment of the invention provides for antimicrobial polymer compositions prepared from the high aspect ratio microcapsules. These compositions are suitable for use as, for example, molding compositions. Articles are prepared from these compositions.

### BRIEF DESCRIPTION OF THE DRAWINGS

Each of FIGS. 1-3 depicts a cross-sectional view of a piece of cut polymer sheet. FIG. 1 is drawn to a much larger scale than either of FIG. 2 or 3, i.e., the antimicrobial particles are about equivalent mean average size yet are depicted as being much smaller in the FIGS. 2 and 3 as compared to FIG. 1.

FIG. 1 represents the prior art wherein the antimicrobial particles are dispersed throughout a non-hydrophilic polymer matrix.

FIG. 2 represents one embodiment of the present invention wherein the high aspect ratio microcapsule is in the form of a fiber or fiber-like structure.

FIG. 3 represents one embodiment of the present invention wherein the high aspect ratio microcapsule is in the form of a flake.

### DETAILED DESCRIPTION OF THE INVENTION

All patent applications, patents, patent publications, and literature references cited in this specification are hereby incorporated by reference in their entirety. In the case of inconsistencies, the present description, including definitions, is intended to control.

According to the present invention, an antimicrobial agent is encapsulated with a hydrophilic polymer and then melt fabricated to form a high aspect ratio microcapsule. The fabrication can be any of several known in the art to include melt spinning to form fibers, and melt casting followed by flaking to form flaked particles. The fibers can be chopped to the desired aspect ratio. Similarly, flaking conditions can be varied to vary the aspect ratio. Optionally, films can be formed followed by slitting, chopping or cutting to the desired aspect ratio. Dependent somewhat upon the fabrication conditions, the aspect ratio may vary from microcapsule to microcapsule. By aspect ratio, we mean the ratio of the longest dimension of a three-dimensioned particle to the shortest dimension. The aspect of a sphere or cube is 1.0. The average aspect ratio of the high aspect ratio antimicrobial microcapsules is typically greater than 2, preferably from about 4 to about 100, and more preferably from about 5 to about 30. Generally, the high aspect ratio antimicrobial microcapsules of the invention include microcapsules that are in the shape of flakes or sheets as well as those that are in the shape of fibers or cylinders. Other shapes, such as football and other oblong shapes, are suitable as well.

While not wanting to be bound by theory, we believe that the encapsulating polymer enables good dispersion of the particle and prevents agglomeration of the antimicrobial agent. Lack of good dispersion necessitates higher loading to impart antimicrobial activity and causes deleterious effects such as poor appearance or decreased physical properties. We believe that the hydrophilic polymer can absorb sufficient water such as to allow slow transport of the metal ion through the encapsulating polymer layer. Consequently, essentially the entire encapsulated antimicrobial agent in high aspect ratio microcapsules touching the surface is available to be transported through the hydrophilic polymer to impart antimicrobial activity. Without this encapsulation, except in the case of hydrophilic coatings and polymers, only the antimicrobial particles at the surface are available to impart activity. The aspect ratio of the microcapsules can be varied dependent upon the application to control the rate of release and therefore be used to impart antimicrobial activity over a sustained period of time. Since the hydrophilic polymer encapsulant enables transport throughout the microcapsule, if any portion of the high aspect ratio microcapsule is at the surface, then the entire antimicrobial agent within that microcapsule is available to impart antimicrobial activity. If the microcapsule is quite long (fiber shaped) then the antimicrobial agent can be separated from the surface by the entire length of the fiber. This results in sustained antimicrobial activity because the transport is done gradually along the entire length of the fiber. Similarly, if the shape is in the form of a flake, if even a portion of the flake is at the surface, then the entire antimicrobial agent within that flake microcapsule is available to impart antimicrobial activity. Everything else being equal, the longer the path of transport, the slower and more sustained the antimicrobial activity. For some end uses, varying rates of release may be desired. This might be desired in applications requiring a high level of initial antimicrobial activity, but then additional antimicrobial activity over a longer period of time. These may be achieved by using high aspect ratio microcapsules of varying aspect ratios.

Additionally, the use of high aspect ratio microcapsules according to the present invention allows one to regulate, to a large extent, the rate of release of the antimicrobial agent. Generally, the rate of release increases as the water absorption of the hydrophilic polymer increases. When used in hydrophilic coatings and polymers, the use of the encapsulated antimicrobial agents allows one to regulate the overall release rate through the polymer coating or matrix. Thus, while a highly hydrophilic polymer matrix would allow for fast water transmission, a lesser hydrophilic polymer used to encapsulate the antimicrobial agent would slow the release of the antimicrobial agent.

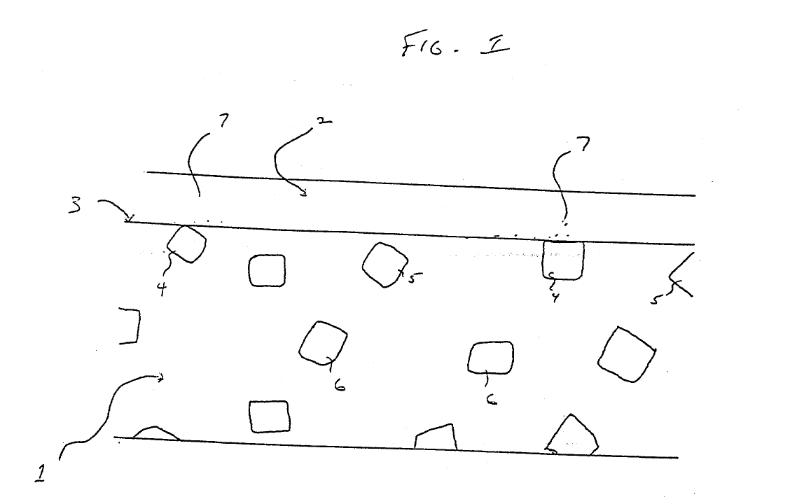

Again, while not wanting to be bound by theory, our understanding can be illustrated by reference to FIGS. 1-3. FIG. 1 represents a cross-sectional view of the prior art system. The polymer matrix (1) has dispersed therein multiple particles of the antimicrobial agent (4, 5 and 6). Only those particles (4) which touch or protrude through the surface (3) are able to release the antimicrobial agent or active (7) into the water film (2). Antimicrobial particles entombed within the polymer matrix (5 and 6), including those close to the surface (5), are incapable of releasing the antimicrobial active since there is no diffusion path to the surface. If water could diffuse through the polymer matrix, it could reach the subsurface particles and access the antimicrobial there. Thus, despite the large deposit of antimicrobial agent in the polymer matrix, the source of efficacious antimicrobial agent is essentially limited to the surface layer of particles having a depth defined by the diameter of the particles. The diffusion kinetics out of those particles is defined by the particle structure, which cannot be altered. The prior art system has no flexibility with regard to source or kinetics, the only parameters we might control.

FIG. 2 depicts a cross-sectional view of one embodiment of the present invention wherein multiple antimicrobial particles (5) are contained in a single hydrophilic high aspect ratio capsule in the form a fiber (8, 9). Here, as with the prior art systems, microcapsules (8) which do not touch the surface (3) are not available to provide antimicrobial efficacy. On the other hand, all of the antimicrobial particles within the fibers touching the surface (9) are available for providing antimicrobial activity. This is due to the transport through the fiber to the surface. The rate of release can be controlled by the hydrophilicity of the encapsulating polymer or by the aspect ratio of the microcapsule.

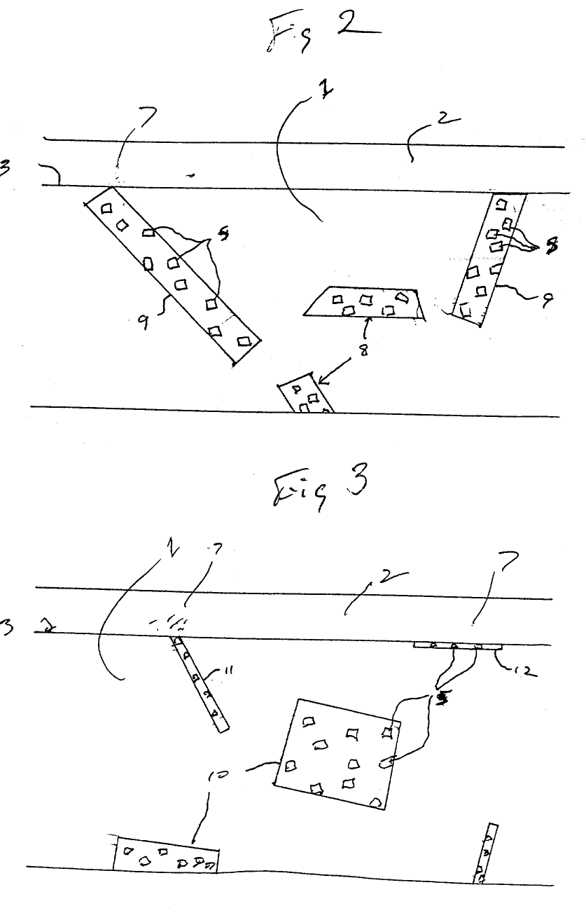

FIG. 3 depicts a cross-sectional view of another embodiment of the present invention wherein multiple antimicrobial particles (5) are contained in a single hydrophilic high aspect ratio capsule in the form a flake (10, 11, 12). Here, as with the prior art systems, microcapsules (10) which do not touch the surface (3) are not available to provide antimicrobial efficacy. On the other hand, all of the antimicrobial particles within the flakes touching the surface (11 and 12) are available for providing antimicrobial activity. This is due to the transport through the flake to the surface. The rate of release can be controlled by the hydrophilicity of the encapsulating polymer or by the aspect ratio of the microcapsule. In the cross-sectional view, microcapsule 12 lies in the plane of the polymer surface (3), microcapsules 10 lay in the plane of the cross-sectional cut and the plane of microcapsule 11 is perpendicular to the cross-sectional cut.

By the term “hydrophilic”, we mean water absorbing, water vapor adsorbing and wettable. By the term “microcapsule”, we mean the antimicrobial agent encapsulated with a hydrophilic polymer. Dependent upon the method used and the desired end use, the amount of particles (5) of antimicrobial agent contained within the microcapsules can be varied from an individual particle of antimicrobial agent to several aggregates of particles of antimicrobial agent. Furthermore, the term “encapsulate” or the phrase “encapsulated within” contemplates that the antimicrobial agent is completely surrounded or encased by hydrophilic polymer as well as that it is substantially surrounded or encased by the hydrophilic polymer. As such, in the latter instance, the antimicrobial agent, in the form of a particle, may touch or protrude from the surface of the microcapsule or a surface of the antimicrobial agent, in the form of a particle, may form part of the surface of the microcapsule. For example, in the case of a flake, the flake may lie in the surface of the microcapsule, i.e., in the surface layer of the microcapsule, so that one entire face of the flake is in direct contact with the matrix polymer and not the encapsulating polymer.

Dependent upon the application, the rate of release can be tailored by proper selection of the hydrophilic polymer used as encapsulant in combination with the choice of the matrix polymer. The high aspect ratio microcapsules should be dispersed in the matrix polymer, but remain as a second phase. Generally, the greater the water absorption of the hydrophilic polymer encapsulant, the greater the rate of release. For a polymer matrix that is highly hydrophilic, the rate of release can be slowed by choice of a hydrophilic polymer encapsulant with a lower water absorption than the matrix polymer.

The choice of the hydrophilic polymer used as an encapsulant will also be determined by the matrix polymer and the method of incorporating the high aspect ratio microcapsules into the matrix polymer. Generally, when the high aspect ratio microcapsules are incorporated by melt compounding with the matrix polymer, the hydrophilic polymer should be chosen such that the high aspect ratio microcapsule will retain a high aspect ratio. If the hydrophilic polymer flows appreciably at a temperature lower than the matrix polymer, then the shape of the microcapsule can be lost during melt compounding or other forming processes. This problem can be avoided by choice of a hydrophilic polymer that does not exhibit too much flow at the processing temperatures. This propensity to flow at processing conditions is principally determined by glass transition for amorphous linear polymers and by melting point for crystalline linear polymers. For crosslinked or highly branched hydrophilic polymers, flow during processing with the matrix polymer does not present the same issues. The foregoing problem is less of an issue with certain types of processing such as for example, compression molding, electrostatic coating, solution coating, reaction injection molding, and pultrusion. If it is desired to use a hydrophilic polymer that does not retain a high aspect ratio upon melt processing with the matrix polymer any of several other methods of incorporation of the high aspect microcapsules can be used rather than melt processing. These methods include, for example, pultrusion, RIM, and powder coating among several others.

Dependent upon the polymer and the processing conditions, prior art antimicrobial agents can cause discolorations. By use of the microcapsules of this invention, any discoloration is limited to the microcapsule and is not throughout the entire polymer matrix. Also, since less antimicrobial agent is necessary with the microcapsule, discoloration is less of a problem.

The antimicrobial agent to be encapsulated contains a metal or metal ion that can impart antimicrobial activity. Examples of such metal ions include silver, copper, zinc, tin, gold, mercury, lead, iron, cobalt, nickel, manganese, arsenic, antimony, bismuth, barium, cadmium, chromium and thallium ions. Metal ions of silver, copper, zinc, and gold are preferred because they are considered safe for in vivo use. Silver ions are more preferred due to the fact that they have the highest ratio of efficacy to toxicity, i.e., high efficacy to low toxicity.

In addition to the metal or metal ion that imparts antimicrobial activity, optionally the antimicrobial agent may include or be used in conjunction with discoloration inhibiting agents and/or dopants. Preferred discoloration inhibiting agents include, but are not limited to inorganic discoloration inhibitors such as those of various ammonium salts. Dopants, which are particularly of use with the ion-exchange type antimicrobial agents, aid in the transport of the antimicrobial metal ion. These dopants provide a ready source of cations, which exchange with and replace the antimicrobial silver metal ions in the ion-exchange ceramic particles, thereby facilitating release and transport of the silver ion. Preferred dopants include, but are not limited to inorganic salts of sodium such as sodium nitrate. For example, if sodium nitrate is used with a silver containing ion-exchange type antimicrobial agent, the sodium nitrate dissociates providing sodium ions which exchange with the antimicrobial silver ions, thereby releasing the silver ion for transport to the surface. In this example, the sodium nitrate expedites the release of the silver from the antimicrobial agent.

The antimicrobial agent can be in the form of a simple salt of the antimicrobial metal such as the oxide, sulfide, chloride, bromide, carbonate, nitrate, phosphate, dihydrogen phosphate, sulfate, oxalate, acetate, benzoate, thiosulfate and the like. Specific examples of suitable salts include silver nitrate, silver oxide, cupric oxide, zinc acetate and zinc oxide.

Alternatively, the antimicrobial agent may be in the form of a water soluble glass containing the antimicrobial agent or compound. Suitable antimicrobial water soluble glasses include those disclose in U.S. Pat. No. 5,470,585. By suitable adjustment of the glass composition, the dissolution rates in water can be controlled. Since their effectiveness requires dissolution in water, they are effective only at the surface where water may be present. The use of high aspect ratio microcapsules of this invention alleviates this problem since the hydrophilic polymer coating can enable water transport to the glass.

Preferably, the antimicrobial agent will be in the form of an ion-exchange type ceramic particle wherein antimicrobial metal ions have been exchanged for (replaced) other non-antimicrobially effective ions in the ceramic particles or a combination of the foregoing with an antimicrobial metal salt. Antimicrobial ceramic particles include, but are not limited to zeolites, hydroxyapatite, zirconium phosphates and other ion-exchange ceramics. Hydroxyapatite particles containing antimicrobial metals are described, e.g., in U.S. Pat. No. 5,009,898. Zirconium phosphates containing antimicrobial metals are described, e.g., in U.S. Pat. Nos. 5,296,238; 5,441,717 and 5,405,644. Because of the two dimensional network structure of zirconium phosphates, delivery of the antimicrobial metal to the surface can be especially difficult. In a zirconium phosphate type particle, the antimicrobial actives are present between sheets of the zirconium phosphate and the passage of the antimicrobial metal ions is limited to the x and y directions defining the space between the sheet, not in the z direction through the sheets themselves. In essence, the zirconium phosphate particle is like an Oreo cookie where the antimicrobial agent is present in the filling. The point of release is at the edges open to the filling, not through the cookies. It is believed that this phenomenon results in somewhat lower efficacy for the zirconium phosphates versus those ion-exchange type antimicrobial particles which release three dimensionally. In this case even zirconium phosphate particles which touch the surface but do not have an open edge touching the surface would not be available to release antimicrobial metal ions. Thus, the use of high aspect ratio microcapsules of this invention alleviates this problem since the metal can be transported through the hydrophilic encapsulant to the surface of the matrix. More preferably, antimicrobial zeolite is employed containing ion-exchanged antimicrobial metal ions.

In antimicrobial zeolite particles used in the preferred embodiment of the present invention, ion-exchangeable ions present in zeolite, such as sodium ions, calcium ions, potassium ions and iron ions are partially replaced with antimicrobial metal ions. Optionally, other ions may also be exchanged for better efficacy and/or color stability, including ammonium ions. Such ions may co-exist in the antimicrobial zeolite particle since they do not prevent the bactericidal effect. Examples of antimicrobial metal ions include, but are not limited to, ions of silver, copper, zinc, gold, mercury, tin, lead, bismuth, cadmium, chromium and thallium. Preferably, the antimicrobial metal ions are silver, copper or zinc ions, and most preferably silver is employed. These antimicrobial metal ions may be incorporated into the zeolite by themselves or in a mixture, for example mixtures of silver and zinc ions or mixtures of silver and copper ions.

The antimicrobial metal ion is present in the range of from about 0.1 to about 25 wt % of the zeolite based upon 100% total weight of zeolite. Preferably, the antimicrobial metal ion is present in the range of from about 0.3 to about 20 wt % of the zeolite based upon 100% total weight of zeolite. Most preferably, the antimicrobial metal ion is present in the range of from about 2 to about 10 wt % of the zeolite based upon 100% total weight of zeolite. In one embodiment, the zeolite contains from about 0.1 to about 15 wt % of silver ions and from about 0.1 to about 15 wt % of copper and/or zinc ions. Although ammonium ion may be contained in the zeolite at a concentration as high as about 20 wt % of the zeolite, it is desirable to limit the content of ammonium ions to about 0.5 to about 2.5 wt % of the zeolite, more preferably from about 0.5 to about 2.0 wt %, and most preferably, from 0.5 to about 1.5 wt %.

Antimicrobial zeolites, including the antimicrobial zeolites disclosed in U.S. Pat. Nos. 4,911,898; 4,911,899 and 4,938,958, are well known and may be prepared for use in the present invention using known methods.

Either natural zeolites or synthetic zeolites may be used to prepare the antimicrobial zeolites used in the present invention. “Zeolite” is an aluminosilicate having a three dimensional skeletal structure that is represented by the formula: XM₂/nO—Al₂O₃—YSiO₂-ZH₂O. M represents an ion-exchangeable ion, generally a monovalent or divalent metal ion; n represents the atomic valency of the (metal) ion; X and Y represent coefficients of metal oxide and silica, respectively; and Z represents the number of water of crystallization. Examples of such zeolites include A-type zeolites, X-type zeolites, Y-type zeolites, T-type zeolites, high-silica zeolites, sodalite, mordenite, analcite, clinoptilolite, chabazite and erionite. The present invention is not restricted to use of these specific zeolites.

The ion-exchange capacities of these zeolites are as follows: A-type zeolite=7 meq/g; X-type zeolite=6.4 meq/g; Y-type zeolite=5 meq/g; T-type zeolite=3.4 meq/g; sodalite=11.5 meq/g; mordenite=2.6 meq/g; analcite=5 meq/g; clinoptilolite=2.6 meq/g; chabazite=5 meq/g; and erionite=3.8 meq/g. These ion-exchange capacities are sufficient for the zeolites to undergo ion-exchange with ammonium and antimicrobial metal ions.

The specific surface area of preferred zeolite particles is preferably at least 150 m²/g (anhydrous zeolite as standard) and the SiO₂/Al₂O₃ mole ratio in the zeolite composition is preferably less than 14 and more preferably less than 11.

The antimicrobial metal ions used in the antimicrobial zeolites should be retained on the zeolite particles through an ion-exchange reaction. Antimicrobial zeolites in which the antimicrobial metal ions are adsorbed or attached without an ion-exchange reaction typically exhibit an overall decreased bactericidal effect and their antimicrobial effect is not long lasting. Nevertheless, it can be advantageous for imparting quick antimicrobial action to maintain a sufficient amount of surface adsorbed metal ion in addition to the ion-exchanged metal ion.

The antimicrobial zeolites, as well as other antimicrobial ceramic particles, may also contain a discoloration agent. Preferably, the discoloration agent is biocompatible. Preferred discoloration agents include, but are not limited to, inorganic discoloration inhibitors such as ammonium. More preferably, the inorganic discoloration inhibitor is an ion-exchanged ammonium ion in the zeolite.

A preferred antimicrobial zeolite for use in the invention is type A zeolite containing a combination of ion-exchanged silver, zinc, copper, and ammonium; silver copper and ammonium or silver and ammonium. One such zeolite is distributed by AgION Technologies, L.L.C. under the product number AW-10N and consists of 0.6% by weight of silver ion-exchanged in Type A zeolite particles having a mean average diameter of about 3μ. Another grade, AJ-10N, consists of about 2% by weight of silver ion-exchanged in Type A zeolite particles having a mean average diameter of about 3μ. Yet another grade, AW-80, contains 0.6% by weight of silver ion-exchanged in Type A zeolite particles having a mean average diameter of about 2μ. Another grade, AJ-80N, consists of about 2% by weight of silver ion-exchanged in Type A zeolite particles having a mean average diameter of about 1μ. These specific zeolites typically contain about 14% by weight zinc in combination with between about 0.5% and 2.5% by weight of ion-exchanged ammonium as a discoloration inhibiting agent.

The hydrophilic polymer used to encapsulate the antimicrobial agent is a polymer that can absorb sufficient water to enable the encapsulated particle to exhibit good antimicrobial behavior, i.e., to allow for the migration and release of the antimicrobial active agent. The polymer will be characterized by having water absorption at equilibrium of at least about 2% by weight measured by ASTM D570. Preferably, the polymer will have water absorption at equilibrium of at least about 5% by weight. More preferably, the polymer will have water absorption at equilibrium of at least about 20% by weight. Especially suitable hydrophilic polymers include those having water contents of from about 50 and to about 150% by weight.

Polymeric compositions for use as the encapsulant in the present invention include polymers, which are comprised of substantial quantities of monomers having polar groups associated with them, such that the overall polymeric composition is rendered hydrophilic. The polar groups can be incorporated into the polymer main chain as in for example polyesters, polyurethanes, polyethers or polyamides. Optionally the polar groups can be pendant to the main chain as in for example, polyvinyl alcohol, polyacrylic acids or as in ionomers such as Surlyn®). Surlyn® is available from Dupont and is the random copolymer poly(ethylene-co-methacrylic acid) wherein some or all of the methacrylic acid units are neutralized with a suitable cation, commonly Na⁺ or Zn⁺². While not being limited by way of theory, it is believed that the inclusion of polar groups allows water to more readily permeate the polymer and consequently, to allow slow transport of the metal ion through the encapsulating polymer layer.

A number of hydrophilic polymers may be used in the present invention and include, for example, (poly)hydroxyethyl methacrylate, (poly)hydroxypropyl methacrylate, (poly)glycerol methacrylate, copolymers of hydroxyethyl methacrylate and methacrylic acid, polyacrylamide, hyaluronan, polysaccharides, polylactic acid, copolymers of lactic acid, (poly)vinyl pyrrolidone, polyamides such as Nylon 6,6 or Nylon 4,6 or Nylon 6,12, cellulosics, polyureas, polyurethanes and certain polyesters containing a high percentage (at least about 10% by weight, preferably at least about 25% by weight or more) of polyalkylene oxide.

The hydrophilic polymer may be a copolymer containing at least a substantial amount of at least one or more of the above-mentioned hydrophilic monomers, including, for example, styrene/methacrylic acid/hydroxyethyl methacrylate copolymers, styrene/methacrylic acid/hydroxypropyl methacrylate copolymers, methylmethacrylate/methacrylic acid copolymers, ethyl methacrylate/styrene/methacrylic acid copolymers and ethyl methacrylate/methyl methacrylate/styrene/methacrylic acid copolymers, copolymers based upon the cellulosics, and copolymers which utilize vinylpyrrolidone monomers, among numerous others.

Other hydrophilic polymers that may be used in the present invention include polyvinyl acetate, polyvinyl alcohol, and copolymers of polyvinyl alcohol and polyvinylacetate, polyvinylchloride, copolymers of polyvinylacetate and polyvinylchloride and hydroxyl-modified vinyl chloride/vinyl acetate copolymers.

Polyurethanes containing a high percentage (at least about 10% by weight, preferably at least about 25% by weight or more) of polyalkylene oxide are especially useful in this invention.

Preferably the hydrophilic polymer is chosen from polyhydroxyethyl methacrylate, polyacrylamide, polyvinylpyrrolidinone, polyurea, polysaccharides, polylactic acid and polyurethane. More preferably, the hydrophilic polymer is hydrophilic polyurethane, such as the TECOPHILIC® polyurethane sold by Thermedics of Woburn, Mass.

The high aspect ratio microcapsules are three dimensional with the longest dimension being generally less than about 3000 microns, preferably from about 5 microns to about 1000 microns, more preferably from about 10 to about 500 microns, most preferably from about 20 to about 100 microns. The average aspect ratio, i.e., the quotient of the longest dimension and the shortest dimension, of the high aspect ratio antimicrobial microcapsules is typically greater than 2, preferably from about 4 to about 100, and more preferably from about 5 to about 30. The longest dimension can be used to regulate the availability and rate of release of the antimicrobial agent. Generally, the longer the high aspect ratio microcapsule, the greater the chance that a portion will touch the surface and make the antimicrobial agent available by transport through the hydrophilic coating. Also the aspect ratio in combination with the hydrophilicity of the hydrophilic polymer coating will determine the rate of release. Generally, longer microcapsules enable continued efficacy over a longer period of time due to the longer transport path of the antimicrobial agent. As disclosed in applicants co-filed application entitled “Encapsulated Inorganic Antimicrobial Additive for Controlled Release”, lower aspect ratio microcapsules, those from 1 to about 4 are also useful and provide certain added benefits, in certain applications, as compared to the higher aspect ratios within the ranges set forth above.

Of course smaller or larger microcapsules can be used. However, the smaller the microcapsule the more closely one approaches a system of individually encapsulated particles. Similarly, the larger the particles, the more potential there is for a deleterious effect on the physical properties of the polymer matrix. Of course, microcapsule size is somewhat dependent upon the size of the antimicrobial particles to be incorporated therein as well as the process by which the microcapsules are made.

Dependent upon the constituents and shape of the high aspect ratio microcapsule, other properties, in addition to antimicrobial activity may also be imparted to the polymer matrix. The effect on the properties will depend upon several factors including the choices of the hydrophilic polymer and the matrix polymer and the method of compounding the high aspect ratio microcapsules into the matrix polymer. Generally, rigid fiber-like microcapsules will impart improved tensile strength and flexural modulus to the polymer matrix. Rigid, flake-shaped particles will generally increase the flexural modulus of the polymer. While the primary benefit of the high aspect ratio microcapsules is the ability to impart antimicrobial activity and to be able to control the rate of release, dependent upon the end use, other factors such as desired modulus may indicate to one skilled in the art the preferred constituents and shape.

The high aspect ratio microcapsule may be prepared by the compounding of the antimicrobial agent with the hydrophilic polymer and fabrication of the compounded product to the desired aspect ratio. This can be done by mixing the antimicrobial agent with the hydrophilic polymer to obtain a blend. The blend is then melt compounded and then fabricated to form a high aspect ratio microcapsule. The fabrication can be any of several known in the art to include melt spinning to form fibers and melt casting followed by flaking to form flaked particles. The fibers can be chopped to the desired aspect ratio. Similarly, flaking conditions can be varied to vary the aspect ratio. Optionally, films can be formed followed by slitting, chopping or cutting to the desired aspect ratio. Dependent somewhat upon the fabrication conditions, the aspect ratio may vary from microcapsule to microcapsule. The fabrication conditions can be varied to obtain an average aspect ratio suitable for the end use application.

The amount of antimicrobial agent encapsulated with the hydrophilic polymer is an amount that is effective to form a microcapsule with good antimicrobial activity. The microcapsules preferably contains from about 1 to about 1000 parts by weight of antimicrobial agent per 100 parts by weight of hydrophilic polymer. More preferably, the microcapsules contain from about 10 to about 200 parts by weight of antimicrobial agent per 100 parts by weight of hydrophilic polymer and, most preferably, from about 20 to about 100 parts by weight of antimicrobial agent per 100 parts by weight of hydrophilic polymer.

The high aspect ratio microcapsules can be used to impart antimicrobial properties to a variety of compositions. They may be blended into various formulations to provide compositions useful for molding, coatings or films with antimicrobial activity. They are particularly useful when blended with polymer formulations to provide compositions useful for molding articles with antimicrobial properties. The combination of hydrophilic polymer encapsulant and matrix polymer should be such that they have a different level of hydrophilicity and such that the microcapsule remains as a second phase in the matrix polymer. The matrix polymer formulations may be based upon either hydrophilic or non-hydrophilic polymers including, but not limited to: polypropylene, polyethylene, polystyrene, ABS, SAN, polybutylene terephthalate, polyethylene terephthalate, nylon 6, nylon 6,6, nylon 4,6, nylon 12, phenolic resins, urea resins, epoxy resins, polyvinylchloride, polyurethanes, silicone polymers, polycarbonates, polyphenylene ethers, polyamides, polyethylene vinylacetate, polyethylene ethyl acrylate, polylactic acid, polysaccharides, polytetrafluoroethylene, polyimides, polysulfones, and a variety of other polymers and copolymers.

The antimicrobial polymer and polymer coating compositions made in accordance with the practice of the present invention may be used in any applications where antimicrobial properties are desirable. Exemplary applications include cutting boards, catheters and other medical devices, pipes, containers, toothbrushes, diapers, air filters, appliances, conveying belts, bottles, liquid dispensers, faucets, humidifiers, air conditioners, mats, razors, and bandages.

The antimicrobial properties of the articles made from the microcapsules of the invention show efficacy for the end use applications. The degree of efficacy may be determined by any of several tests such as the Dow shaker test, direct inoculation and several others known to those skilled in the art and chosen based upon the end use application.

The present invention will hereunder be explained in more detail with reference to the following non-limiting working examples.

### EXAMPLES

### Example 1

A polymer blend is made by first compounding an antimicrobial agent with a hydrophilic polymer and then using the high aspect ratio microcapsule in a polymer formulation. Fifty parts by weight of AgION AJ10D (about 2.5% by weight of silver ion-exchanged in Type A zeolite particles having a mean average diameter of about 3μ) is gravimetrically fed with 50 parts by weight of Tecophilic®) 60 resin, a hydrophilic polyurethane available from Thermedics Inc. having a moisture absorption of about 60%. The blend is extruded using a Leistriz 27 mm twin screw extruder through a holed die to produce strands which are fed into a pelletizer to produce conventional pellets. The pellets are then fed into a fiber extruder equipped with a chopper to produce finely chopped fibrous microcapsules with average dimensions of 20 microns in diameter and 500 microns in length resulting in an aspect ratio of 25. A mixture of 4 parts by weight of these high aspect ratio microcapsules in the shape of fibers is combined with 96 parts by weight of LDPE, blended by shaking and injection molded into 5 cm by 5 cm by 0.16 cm test parts. The injection molding temperature conditions are 385° F. rear zone; 390° F. front zone and 395° F. nozzle temperature. The test parts are expected to exhibit good antimicrobial activity.

### Example 2

A polymer blend is made by first compounding an antimicrobial agent with a hydrophilic polymer and then using the high aspect ratio microcapsule in a polymer formulation. Fifty parts by weight of AgION AJ10D (about 2.5% by weight of silver ion-exchanged in Type A zeolite particles having a mean average diameter of about 3μ) is gravimetrically fed with 50 parts by weight of Tecophilic® 60 resin, a hydrophilic polyurethane available from Thermedics Inc. having a moisture absorption of about 60%. The blend is extruded using a Leistritz 27 mm twin-screw extruder and pelletized. The pellets are then ground to a powder. The powder is then fed into a thermal spray system that blows the powder through a flame, melting the particles. The spray of molten polymer is directed onto a cooled, low-adhesion drum under conditions that the particles do not melt together into a solid coating, but stay as individual flattened particles on the drum. The size of the flakes, determined by the size of the starting particles, is about 100 microns in diameter and 10 microns thick resulting in an aspect ratio of 10. The drum also has a scraping bar that removes the flake-like particles. A mixture of 4 parts by weight of these high aspect ratio microcapsules in the shape of flakes is combined with 96 parts by weight of LDPE, blended by shaking and injection molded into 5 cm by 5 cm by 0.16 cm test parts. The injection molding temperature conditions are 385° F. rear zone; 390° F. front zone and 395° F. nozzle temperature. The test parts are expected to exhibit good antimicrobial activity.

### Example 3

Pellets are prepared as in Example 1 and are then fed into a fiber extruder equipped with a chopper to produce finely chopped fibrous microcapsules with average dimensions of fiber length of 150 microns and diameter of 25 microns to result in an aspect ratio of 6. The high aspect ratio microcapsules in the shape of fibers are blended by agitation with powder coating powder and electrostatically sprayed onto a surface. The coated part is then heated to fuse the coating. In the resulting structure, the fibers are oriented perpendicular to the treated surface with the ends exposed to release silver. The structure is expected to exhibit good antimicrobial activity.

Thus the high aspect ratio microcapsules of the invention are exceptionally suitable for use in various compositions and provide compositions without the deleterious effects of the prior art.

While preferred embodiments of the invention have been described in the foregoing examples, it will be understood by those skilled in the art that various changes and modifications may be made therein without departing from the spirit and the scope of the invention. Accordingly, the above description should be construed as illustrating and not limiting the scope of the invention.

What is claimed is:

1. A high aspect ratio microcapsule comprising an antimicrobial agent coated with a hydrophilic polymer, said microcapsule having an aspect ratio of greater than about 2.

2. A high aspect ratio microcapsule according to claim 1 wherein the inorganic antimicrobial agent contains a metal or metal ion selected from the group consisting of silver, copper, zinc, tin, gold, mercury, lead, iron, cobalt, nickel, manganese, arsenic, antimony, bismuth, barium, cadmium, chromium, thallium and combinations thereof.

3. A high aspect ratio microcapsule according to claim 2 wherein the antimicrobial metal or metal ion is silver, zinc, copper or a combination of any two or all three of the foregoing.

4. A high aspect ratio microcapsule according to claim 1 wherein the antimicrobial agent is selected from the group consisting of metal salts, metal oxides, antimicrobial water soluble glasses, antimicrobial metal ion-exchange type agents and combinations thereof.

5. A high aspect ratio microcapsule according to claim 4 wherein the antimicrobial agent is an antimicrobial metal ion-exchange type agent comprising a ceramic carrier having ion-exchanged antimicrobial metal ions.

6. A high aspect ratio microcapsule according to claim 5 wherein the ceramic carrier is selected from the group consisting of zeolites, hydroxyapatites and zirconium phosphates.

7. A high aspect ratio microcapsule according to claim 6 wherein the antimicrobial agent is a zeolite that contains silver ions.

8. A high aspect ratio microcapsule according to claim 1 wherein the hydrophilic polymer is a polymer with water absorption at equilibrium of at least about 2% by weight.

9. A high aspect ratio microcapsule according to claim 8 wherein the hydrophilic polymer is a polymer with water absorption at equilibrium of at least about 5% by weight.

10. A high aspect ratio microcapsule according to claim 9 wherein the hydrophilic polymer is a polymer with water absorption at equilibrium of at least about 20% by weight.

11. A high aspect ratio microcapsule according to claim 10 wherein the hydrophilic polymer is chosen from polyhydroxyethyl methacrylate, polyacrylamide, N-vinyl-2-pyrrolidinone, polysaccharides, polylactic acid, polyamide and polyurethane.

12. A high aspect ratio microcapsule according to claim 11 wherein the hydrophilic polymer is polyurethane.

13. A high aspect ratio microcapsule according to claim 1 wherein the microcapsule contains from about 1 to about 1000 parts by weight of antimicrobial agent based upon 100 parts by weight of hydrophilic polymer.

14. A high aspect ratio microcapsule according to claim 13 wherein the microcapsule contains from about 10 to about 200 parts by weight of antimicrobial agent based upon 100 parts by weight of hydrophilic polymer.

15. A high aspect ratio microcapsule according to claim 14 wherein the microcapsule contains from about 20 to about 100 parts by weight of antimicrobial agent based upon 100 parts by weight of hydrophilic polymer.

16. A high aspect ratio microcapsule according to claim 1 further comprising an inorganic discoloration inhibiting agent.

17. A high aspect ratio microcapsule according to claim 16 wherein said discoloration inhibiting agent is an ammonium compound.

18. A high aspect ratio microcapsule according to claim 17 wherein the antimicrobial agent comprises an ion-exchange type antimicrobial agent and said inorganic discoloration agent comprises ion-exchanged ammonium ions contained within said antimicrobial agent.

19. A high aspect microcapsule according to claim 1 further comprising a dopant agent.

20. A high aspect ratio microcapsule according to claim 19 wherein said dopant is an inorganic sodium salt.

21. A microcapsule according to claim 20 wherein said dopant is sodium nitrate.

22. A high aspect ratio microcapsule according to claim 1 wherein the high aspect ratio microcapsule comprises multiple particles of one or more antimicrobial agents encapsulated within a hydrophilic polymer.

23. A method for manufacture of a high aspect ratio microcapsule comprising:
(a) compounding of the antimicrobial agent with the hydrophilic polymer
(b) fabricating the compounded product to the desired size and aspect ratio.

24. The method of manufacture of a high aspect microcapsule according to claim 23 wherein the antimicrobial agent is a zeolite containing silver.

25. A method for manufacture of a high aspect ratio microcapsule according to claim 23 wherein the compounding is done by a method selected from the group of extrusion, pultrusion, RIM and powder coating.

26. A method for manufacture of a high aspect ratio microcapsule according to claim 25 wherein the compounding and fabrication is done by extrusion into fibers and chopping the fibers to the desired length.

27. A method for manufacture of a high aspect ratio microcapsule according to claim 23 wherein the fabrication is done by flaking.

28. A method for manufacture of a high aspect ratio microcapsule according to claim 23 wherein the fabrication is done by preparation of a film and chopping the film to the desired size.

29. A method for manufacture of a high aspect ratio microcapsule according to claim 23 wherein the fabrication is done to give high aspect ratio microcapsules wherein the average longest dimension is less than about 3000 microns.

30. A method for manufacture of a high aspect ratio microcapsule according to claim 29 wherein the fabrication is done to give high aspect ratio microcapsules wherein the average longest dimension is from about 5 microns to about 1000 microns.

31. A method for manufacture of a high aspect ratio microcapsule according to claim 30 wherein the fabrication is done to give high aspect ratio microcapsules wherein the average longest dimension is from about 10 microns to about 500 microns.

32. A method for manufacture of a high aspect ratio microcapsule according to claim 31 wherein the fabrication is done to give high aspect ratio microcapsules wherein the average longest dimension is from about 20 microns to about 100 microns.

33. A high aspect ratio microcapsule according to claim 1 wherein the aspect ratio is from about 4 to about 100.

34. A high aspect ratio microcapsule according to claim 33 wherein the aspect ratio is from about 5 to about 30.

35. A polymer composition comprising the high aspect ratio microcapsule of claim 1 and a matrix polymer wherein the high aspect ratio microcapsule comprises a discrete phase within the matrix polymer.

36. The polymer composition of claim 35 wherein the matrix polymer is an addition polymer selected from the group consisting of polypropylene, polyethylene, polystyrene, polyvinylchloride, ABS, SAN, epoxy resins and polytetrafluoroethylene,

37. The polymer composition of claim 35 wherein the matrix polymer is a condensation polymer selected from the group consisting of polyesters, polycarbonates polyurethanes, polyimides, polyamides and silicone polymers.

38. The polymer composition of claim 35 wherein the matrix polymer is not a hydrophilic polymer.

39. The polymer composition of claim 35 wherein the matrix polymer is a hydrophilic polymer whose hydrophilic property is different from that of the hydrophilic polymer encapsulant used to make the high aspect ratio microcapsule.

40. The polymer composition of claim 35 wherein the matrix polymer is a copolymer.

41. The polymer composition of claim 35 wherein the matrix polymer is a polymer blend.

42. A method of preparing an antimicrobial resin comprising incorporating a high aspect ratio antimicrobial microcapsule according to claim 1 into a polymer matrix wherein the polymer matrix is not the same polymer as used to form the high aspect ratio microcapsule.

43. The method of claim 42 wherein the high aspect ratio microcapsule is melt blended with the polymer matrix material.

44. The method of claim 42 wherein the high aspect ratio microcapsule is dry blended with a second polymer in powder form and powder coated onto a substrate.

45. A method of controlling the rate of release of an antimicrobial agent from a polymer matrix comprising forming a high aspect ratio microcapsule comprising an antimicrobial agent encapsulated within a hydrophilic polymer of a given hydrophilic property which allows for the release of the antimicrobial agent at a given rate and incorporating the high aspect ratio antimicrobial microcapsule into another polymer which is either non-hydrophilic or which has a different hydrophilic property.

46. A method of improving the antimicrobial properties of a non-hydrophilic polymer using a given amount of an antimicrobial agent comprising forming a high aspect ratio microcapsule comprising the antimicrobial agent encapsulated within a hydrophilic polymer and incorporating the antimicrobial microcapsule into the non-hydrophilic polymer to form an antimicrobial composition having improved antimicrobial performance as compared to a similar composition wherein the antimicrobial agent is directly incorporated into the non-hydrophilic polymer.
